(19) United States
(12) Patent Application Publication
(10) Pub. No.: US 2024/0076616 A1
(43) Pub. Date: Mar. 7, 2024

(54) METHOD FOR T-CELL EXPANSION AND RELATED MEDICAL APPLICATIONS

(71) Applicant: Cytovac A/S, Hørsholm (DK)

(72) Inventor: Alexei Kirkin, Hørsholm (DK)

(73) Assignee: Cytovac A/S, Hørsholm (DK)

(21) Appl. No.: 18/262,268

(22) PCT Filed: Jan. 21, 2022
(86) PCT No.: PCT/EP2022/051310

§ 371 Date: Jul. 20, 2023

(30) Foreign Application Priority Data
Jan. 21, 2021 (EP) 21152817.9

(51) Int. Cl. C12N 5/0783; A61K 35/17; C12N 5/00

(52) U.S. Cl. CPC C12N 5/0636; A61K 35/17; C12N 5/0018; C12N 2501/02; C12N 2501/22; C12N 2501/2301; C12N 2501/2302; C12N 2501/2304; C12N 2501/2306; C12N 2501/2312; C12N 2501/25; C12N 2502/1121

(57) ABSTRACT

Provided is a method for preparation of a composition comprising activated human CD4+ and CD8+ lymphocytes. The method entails use of allogeneic mature dendritic cells as feeder cells added at an early stage in the induction of proliferation and activation of CD4+ and CD8+ T cells. Further provided is a method for treatment of lymphopenia related diseases by infusion of the cells obtained from the present process.

### FIELD OF THE INVENTION

The present invention relates generally to adaptive immunotherapy. More specifically, the present invention relates to methods for acceleration production of large numbers of mature activated T_{H} and cytotoxic T cells that are useful in adaptive immunotherapy and also relates to the medical application of T-cells thus produced.

### BACKGROUND OF THE INVENTION

Many severe diseases cause lymphocytopenia or lymphopenia.

In particular, a reduced number of CD4⁺ T cells (T_{H} cells) and CD8⁺ cytotoxic T cells in the blood is a hallmark of infection with many potentially lethal viruses such as: SARS-CoV, SARS-CoV-2, MERS-CoV. Also, infections causing viral hepatitis and bacterial infections such as tuberculosis and typhoid fever infections in humans (Levine et al. 2002; Zhang et al. 2020; Boonnak et al. 2014; www.nhlbi.nih.gov/) share this hallmark. Since T_{H} cells are central players in activating other cell types in the immune system, which eradicate virus and other pathogens, the reduced number of these cells constitute a serious problem for the patient. The CD8⁺ cytotoxic lymphocytes and natural killer cells are direct able to kill virus infected cells and thereby overcome the viral infection

The three types of human lymphocytes are T lymphocytes (T-cells), B lymphocytes (B-cells), and natural killer cells (NK cells). All three cell types protect the body against infection. Most people who suffer from lymphocytopenia exhibit low numbers of T lymphocytes.

Lymphocytopenia is a result of either destruction of lymphocytes as can be observed in HIV patients, impaired proliferation of new lymphocytes, retention of lymphocytes in the spleen or lymph nodes, or a combination of these factors.

In healthy humans the number and composition of T cells in the blood are tightly controlled at a relatively constant level throughout life (Kumar et al. 2018). T cell homeostasis requires a balance between apoptosis and proliferation. Alterations in the homeostasis trigger a series of compensatory mechanisms that aim to reinstate homeostatic equilibrium after the infection is cleared.

Dysregulation in T cell homeostasis is part of the pathology in several diseases caused by virus and bacteria, including infections with SARS-CoV-2, influenza, HIV, SARS, and MERS, viral hepatitis, tuberculosis, and typhoid fever. The severity of lymphocytopenia is correlated with the mortality rate in severe COVID-19 and influenza infections (Zhang et al. 2020; Boonnak et al. 2014).

Within T cell immunotherapy technology, Chimeric antigen receptor (CAR) T cell therapy against leukaemia is commercially available and on the market: Kymriah (tisagenlecleucel) and Yescarta (axicabtagene ciloleucel). On the other hand, no anti-viral cellular immunotherapy against COVID-19 or other viral diseases is commercially available at this time point.

Traditionally, mitogenic lectins such as phytohemagglutinin (PHA) and concanavalin A (Con A) have been used for polyclonal T cell stimulation. A more physiologically relevant approach uses beads coated with anti-CD3 and anti-CD28 to stimulate T cells in a manner that partially mimics stimulation by antigen-presenting cells. Anti-CD3/CD28 antibodies have been used for ex vivo expansion of autologous T lymphocytes for treatment of several diseases such as HIV infection. Adoptive transfer of isolated and activated CD4⁺ T_{H} cells leads to expansion of peripheral T cells, preferentially CD4⁺ cells, in HIV infected individuals without sign of significant toxicity (Levine et al., 2002). Importantly, virus load was significantly decreased during in vitro expansion of lymphocytes, and there was no increase in virus load after injection of lymphocytes to the patients. The authors suggested that the increase in number of CD4⁺ T cells was mediated by indirect mechanisms, probably associated with the ability of the injected cells to secrete cytokines, in particularly GM-CSF, that was previously shown to be able to increase the level of CD4⁺ T cells in HIV infected patients (Brites et al, 2000). Injection of autologous activated lymphocytes was also shown to restore immunological reactivity of T lymphocytes. Restoration of the number of lymphocytes and their immunological reactivity was demonstrated in two other indications with chemically induced lymphopenia: in paediatric patients with neuroblastoma (Grupp et al, 2012), and in patients with non-Hodgkin lymphoma (Laport et al, 2003). The CD3/CD28 antibody-based protocol for lymphocyte expansion is currently used for preparation of CAR-T cells. The principal disadvantage of the commonly used CD3/CD28-based expansion protocol is the high variability in properties of the expanded lymphocytes, as well as a low proportion of cells with the central memory phenotype which are required for the effective clinical effect of the transferred lymphocytes. This may partly be attributed to the damaging effect of these antibodies to the lymphocytes. Indeed, up to 40% death of CD8⁺ T cells, and up to 20% death of CD4⁺ T cells has been described by Laux et al., 2000.

Van den Bergh J. M. J. et al. 2017, Cancer Immunology Research 5(8): 710-715, discloses genetically modified, monocyte-derived dendritic cells, which are tested in vitro for their suitability as a vaccine agent in cancer patients to enhance graft-versus-tumor responses in connection with allogeneic stem cell transplantation. The genetically modified dendritic cells were tested in an allogeneic mixed lymphocyte reaction for their ability to stimulate T lymphocytes to proliferate; using this approach, 50%-60% of CD4⁺ and 60%-70% of CD8⁺ lymphocytes were shown to proliferate.

### OBJECT OF THE INVENTION

It is an object of embodiments of the invention to provide an expedient and rapid method for generation of activated, proliferating human T lymphocytes, said T lymphocytes being suitable for treatment of lymphopenia. It is a further object of the invention to provide such a treatment of lymphopenia.

### SUMMARY OF THE INVENTION

The T cell reconstitution immunotherapy described disclosed herein is based in part on experience obtained when carrying out a well-established anti-cancer immunotherapy technology platform (“ALECSAT”, cf. Kirkin et al. 2018 as well as WO 2020/208054).

In brief, the ALECSAT technology includes in the early steps that proliferating autologous CD4⁺ T-cells are expanded by co-culture of autologous lymphocytes with autologous mature dendritic cells (autoDCs). Subsequently, the CD4⁺ T-cells are brought to express cancer/testis antigens and thereafter they are used for “immunization” of yet a batch of autologous T-cells, leading ultimately to the provision of a T-cell population, which is enriched for CD8⁺ and NK cells that are injected back into a patient to treat a cancer. In this process, it is important that all cell populations used are autologous because non-autologous cells increase the risk of inducing undesired and potentially fatal immune responses in the patients to whom the activated cells are administered. A further problem of using non-autologous dendritic cells, in particular in the most recently developed protocols of the ALECSAT technology where dendritic cells are used as feeder cells late in the process during the culture of the proliferating CD4⁺ cells and autologous lymphocytes, is that non-autologous cells are recognized as foreign and therefore attacked and killed by the very T-cells they are supposed to support during proliferation; use of non-autologous dendritic cells would hence be counterproductive in the ALECSAT process.

The present invention presents a highly simplified and optimized process compared to the ALECSAT technology when evaluating on several parameters. First of all, it has been found that application of allogeneic dendritic cells as feeder cells for autologous T cells during the early-stage process will lead to increases in the total numbers of activated CD4⁺ and CD8⁺ T-cells obtained when compared to the situation where use is made of autologous cells.

Since it takes 6 days to initially produce fully mature autologous dendritic cells from a patient and thereafter 7 days to obtain proliferating T-cells after co-culture of lymphocytes with the mature dendritic cells (and then further 13 days to manufacture the final T-cell population), CD4⁺ cells obtainable in the ALECSAT process are not suited for treatment of patients suffering from acute lymphocytopenia.

In contrast, it has been found by the present inventors that the protocol presented herein can manufacture cells useful for immunotherapy cells within only 7 days, as a consequence of the use of allogeneic dendritic cells, because these can be produced in advance and cryopreserved (see FIG. 1).

The manufacturing protocol for the new immunotherapy cells (called SurviveVirus herein) is based on the discovery of the efficiency of adding allogeneic mature dendritic cells (alloDCs) to the culture of T-cells.

Allogeneic mature dendritic cells have herein been shown to exhibit two major advantages compared to autologous dendritic cells (autoDCs) which are used in the ALECSAT process (FIG. 2):
Firstly, alloDCs are herein demonstrated to stimulate proliferation of T cells more effectively than autologous DC's (see Table 1 below), which can be observed as a higher growth rate. The T-cell number increases 38% more when stimulated with alloDCs when compared to stimulation with autoDCs during 7 days of co-culture (FIG. 2). The increase is with the current sample size not sufficiently large to be significant (P=0.1) but nevertheless marked.
Secondly, the alloDC's can advantageously be manufactured in advance and cryopreserved, thus cutting the protocol for manufacturing of SurviveVirus cells 6 days shorter than if utilising autoDCs produced from the patient's own monocytes.

In addition to this, it is noted that it in many practical settings is highly advantageous (or even of essence) that the immunotherapeutic CD4⁺ cells can be manufactured within only 7 days from the date of obtaining blood from the patient. This short time span allows the cell population to be administered to the patient before the lymphocytopenia turns out to be lethal.

SurviveVirus cells are manufactured with alloDCs as feeder cells from a healthy donor and is therefore per definition not an autologous immunotherapy as the ALECSAT immunotherapy. The alloDCs stimulate the proliferation and activation of the patients' (subject's) T cells. The activated T cells are able to recognize the alloDCs as foreign and kill approximately 90% of them during the 7 days it takes to manufacture SurviveVirus cells (Table 2). The final SurviveVirus immunotherapeutic cells which are infused into the patient contain 0.02% allogeneic cells. So, even though alloDCs have been applied to stimulate the proliferation and activation of patient T cells during the manufacturing of the immunotherapy SurviveVirus, the cells infused back into the patient are 99.98% autologous since the majority of the alloDCs have been killed during the last stage of cell culture. This means that the infused immunotherapy only consists of autologous lymphocytes and a little fraction of alloDC's, which is considered a safe therapy, because alloDC's are not able to induce graft-versus-host (GvHD).

In the situation where allogeneic dendritic cells have been used to stimulate the proliferation of T cells, have we investigated the amount of allogeneic dendritic cells which are left in the cell culture after 7 days co-culture. In two cultures from donors 109/20 and 111/20 which were both HLA-A2 negative, were the allogeneic dendritic cells added from an HLA-A2 positive donor (110/20 and 112/20) respectively. In both cases were the amount of allogeneic dendritic cells (CD83⁺ HLA-A2⁺) measured in the final drug product after 7 days co-culture. In both batch 109 and 111 were one allogeneic cell found within a cell sample of 5000 FACS analyzed cells (Table 2).

This means that approximately 89600 allogeneic dendritic cells are left in the final drug product produced with cells from donor 109/20. Which is approximately 8.96% of the 1 million allogeneic dendritic cells which were added to the co-culture day 0. For the batch produced on cells from donor 111/20 were approximately 102400 allogeneic dendritic cells left in the final drug product, which is approximately 10.2% of the added amount. During the co-culture will the cytotoxic CD8 positive T cells recognize the allogeneic dendritic cells as foreign and start to kill them.

Safety of immunotherapy with allogeneic dendritic cells has been studied in a phase 1 clinical trial where patients received four biweekly intradermal allogeneic dendritic cell injections at doses up to 50 million cells. It was concluded that allogeneic dendritic cell injections in patients was safe, feasible and generates both cellular and humoral immune responses (Loosdrecht et al. 2018). The Efficacy and Safety is now studied in a Phase II study (ClinicalTrials.gov Identifier: NCT03697707).

Another allogeneic DC-based immunotherapy using pre-produced and freeze-stored DCs from healthy blood donors as an off-the-shelf immune enhancer, induced no severe adverse events in the patients (Laurel) et al. 2017). The use of MHC-incompatible allogeneic DCs will further induce a local rejection process at the injection site that is expected to further enhance recruitment and maturation of endogenous bystander DCs.

It is our assessment that the relatively small amount of allogeneic dendritic cells left in the therapeutic preparation is not a danger to the patient receiving the SurviveVirus immunotherapy. These remaining allogeneic DCs left in our preparation will not be increasing in number after injection because DC's are not proliferating cells, and should be soon eliminated due to the patient's development of the immune response against them. Due to low number of the injected DCs this immune reaction will be of very low intensity, and will not lead to induction of cytokine storm.

12 cultures were produced from peripheral blood from 6 donors, with starting number of 10 million lymphocytes (day 0). In average 493 (SD 149) million cells were produced in co-culture with allogenic dendritic cells. In the cultures with autologous dendritic cells as feeder cells were the average cell number 356 (SD 139) million cells, which is 38% less than in the allogeneic situation (FIG. 2). The difference is noticeable but not statistically significant with the current sample numbers (p=0.1). This means the original number of cells has expanded 49.3 and 35.6 times during the 7 days in co culture with the allogenic and autologous dendritic cells respectively.

The autologous cell culture consists in average of 96.3% T cells (CD3⁺), among them are 80.5% CD4⁺ T helper cells and 15.6% CD8⁺ cytotoxic T cells. The allogenic cell culture consists in average of 97.4% T cells (CD3⁺), among them are 58.8% CD4⁺ T helper cells and 38.5% CD8⁺ cytotoxic T cells (Table 3). Both cell cultures stimulated by allogenic and autologous feeder cells contained more than 96% T cells. The amount of CD4⁺ T helper cells is significantly higher in the situation where the cell culture is stimulated by autologous dendritic cells (p=0.004). Opposite is the amount of CD8⁺ cytotoxic T cells significantly higher in the situation where the cell culture is stimulated by allogeneic dendritic cells (p=0.003) (FIG. 4).

The final composition of SurviveVirus cells thus consists of approximately 58.8% CD4⁺ T cells and 38.5% CD8⁺ cytotoxic T cells (see Table 3). The final cell composition of SurviveVirus become more similar to ALECSAT cells when alloDCs are applied compared to autoDCs. It is important that the cell composition is comparable between SurviveVirus and ALECSAT, since the beneficial effect against lymfocytopenia in patients has been observed with infusion of ALECSAT cells in cancer patients (FIG. 3).

In the ALECSAT immunotherapy the T cells are immunized against cancer antigens. The T cells in SurviveVirus products are manufactured without priming against specific antigens from pathogens or cancer.

When adding dendritic cells to T cell cultures, the proliferation rate of especially the T_{H} cells exponentially increases, and the T cells get activated. This is important for their ability to subsequently stimulate other cell types in the immune system when the T_{H} cells are infused back into the patient. The activation of the T cells is seen as changed phenotype characteristics such as expression of other kinds of cluster differentiation (CD) markers on their cell surface, changed cytokine expression profiles and increased cell diameter from approximately 8.5 μm to 12.4 μm (Table 3). The cytokine profile of the SurviveVirus cells has been investigated intensively in order to make sure that it is safe to apply SurviveVirus cells to patients who already have increased level of proinflammatory cytokines.

Interferon-γ (IFN-γ) is produced by CD4 and CD8 positive T cells and the function of IFN-γ is to activate other immune cells such as macrophages. Macrophages secretion of IL-6 is the mechanism behind cytokine storm in severe COVID-19 patient (Zhang et al. 2020). We have measured the level of IFN-γ in the cell media when SurviveVirus cells were harvested. There is no difference in the IFN-γ level whether the batch is produced with Allo- or AutoDC's, and in general is the IFN-γ level low (FIG. 5). The average IFN-γ is 188 (+/−) 61.5 pg/mio cells which is the same level as produced by ALECSAT cells (Kirkin et al 2018) which has demonstrated high level of safety in 6 clinical trials without induction of cytokine storm.

To conclude, by producing proliferating, activated T-cells according to the present invention's protocol using alloDCs as feeder cells, it is possible to supply pharmaceutically acceptable and immune therapeutically effective T-cells to lymphocytopenia patients within a time-span of only 7 days. The T cells obtained are not antigen specific and can be applied against e.g. SARS-CoV-2 causing COVID-19 as well as front-line T cell treatment against novel viral diseases in the future or to patients hospitalized with other diseases causing severe lymphocytopenia.

Finally, and importantly, the administration to patients of the T-cells obtained according to the invention appear to entail the highly unexpected advantage of dramatically increasing the patient's T-cell numbers (in particular the CD4⁺ T-cell numbers) to a degree which far supersedes the number of T-cells administrated (see FIG. 3 and explanation below). In other words, the patients receiving the therapy will benefit not only from the T-cell increase provided by the administration of the T-cells, but also and importantly from a rapid mobilisation of their own T-cells, and in particular their CD4⁺ cells. The present inventors have already demonstrated efficiency of a similar immunotherapy against lymphocytopenia, in treatment of patients with relapsed glioblastoma also suffering from lymphocytopenia (Kirkin et al., 2018). Further investigation of this discovery surprisingly demonstrated that 5 out of 5 patients who received one injection of the therapeutic immunotherapy ALECSAT returned to normal level (400-1600 cells/μl) of CD4⁺ T cell in the blood within 24 hours (FIG. 3). The observed fast increase in CD4⁺ T cells in the blood (FIG. 3) is not only due to the number of infused T cells but is believed to be due large endogenous mobilisation of T helper cells from the patient's bone marrow. The mechanism behind the observed release of CD4⁺ T helper cells to the bloodstream is related to the infusion of the mature dendritic cell stimulated and activated autologous T cells, but the exact underlying molecular mechanism(s) is/are unknown.

Infusions with SurviveVirus cells containing high numbers of activated autologous T cells is therefore believed to have the same effect on lymphocytopenia, as demonstrated for ALECSAT infusions, because the cell numbers and cell composition is very similar.

Currently, the main procedure used to upscale the production of T lymphocytes is the prior art method described for ALECSAT preparation (Kirkin et al., 2018). The principal element of this procedure is use of feeder cells, consisting of mature dendritic cells derived from autologous monocytes. The disadvantages of using autologous mature dendritic cells as described according to the existing protocol for expansion of cytotoxic lymphocytes, is that it prolongs the protocol with the 6 days it takes to generate mature dendritic cells. It is possible to generate autologous mature dendritic cells faster than 6 days (Dudley et al 2003), but they do not stimulate the proliferation of the T cells as effectively, thus resulting in the final T cell composition expressing high level of inhibitory molecules like PD1 on their surface.

So, in a first aspect the present invention relates to a method for preparation of a composition of human T-cells, said composition comprising proliferating and activated CD4⁺ and CD8⁺ cells, the method comprising a) mixing mononuclear cells from a human subject with mature allogeneic human dendritic cells, b) co-culturing the mixed cells from step a) under conditions that stimulate proliferation of CD4⁺ and CD8⁺ T lymphocytes, whereby the lymphocyte number is increased and the lymphocyte phenotypes are altered, and c) harvesting and optionally isolating T lymphocytes from the co-culture no later than 7 days after step a.

In a second aspect the present invention relates to a method for supplementing a human subject with lymphocytes, such as in a treatment of lymphocytopenia in a human subject, the method comprising obtaining a sample comprising blood cells from the subject, isolating mononuclear cells from the sample and subsequently preparing a composition of human T-cells according to the method of the first aspect of the invention and any embodiments thereof, wherein the mononuclear cells in step a are the mononuclear cells from the sample, and subsequently administering an effective amount of the T lymphocytes obtained from step c to the patient.

In a third aspect, the present invention relates to a method for treatment of lymphocytopenia in a human subject, the method comprising administering to said human subject T-lymphocytes obtained by the method of the first aspect of the invention and any embodiments thereof, wherein said T-lymphocytes are obtained from the same said human subject.

A fourth aspect of the invention relates to a composition of proliferating, activated T-lymphocytes obtainable or obtained by the method according to the first aspect of the invention and any embodiments thereof. In two related aspects the invention relates to this composition for use as a medicament and for use in the treatments of the 2^{nd} and 3^{rd} aspects of the invention and any embodiments thereof.

### LEGENDS TO THE FIGURE

FIG. 1: Schematic outline of a process of the invention for preparation of activated, proliferating T-lymphocytes.

Manufacturing of the cells is divided into two separate processes. Process 1 outlines the 6-day production process generating mature dendritic cells from the healthy donor monocytes.

Step 1 is separation of peripheral blood mononuclear cells (PBMC) from donor blood or leukapheresis product. Step 2: Monocyte purification on column. Step 3: Generation of allogeneic monocyte derived mature dendritic cells.

Process 2 takes 7 days if frozen allogeneic mDC's is produced in advance (via process 1).

Step 1 is separation of peripheral blood mononuclear cells (PBMC) from patient blood or leukapheresis product. Step 2: Mixing of allogeneic mDC's and autologous lymphocytes 1:10. Step 3: The mDC's induce activation and exponential growth of the CD4⁺ and CD8⁺-enriched lymphocytes. Step 4: The activated T cells are harvested analysed and infused into the patient.

FIG. 2: Graph showing increase in cell numbers during co-culture of mature DCs and PBMC. There is no statistically significant difference between the numbers of produced cells whether allogeneic or autologous mature DCs are used as feeder cells (p=0.1).

FIG. 3: Graph showing increase in CD4⁺ T-lymphocytes in patient blood after infusion of autologous activated, proliferating T-lymphocytes.

A large increase in circulating T helper lymphocytes in the blood of five randomly selected glioblastoma multiforme patients 1 and 2 days after infusion of the immunotherapy ALECSAT described above. The total numbers of CD4⁺ lymphocytes in the patients' blood were measured immediately before and 1 and 2 days after infusion of the immunotherapy. The increase in CD4⁺ T helper lymphocytes observed in the patient's blood after each treatment cannot be explained by the number of infused T cells (median 6.8×10⁷ cells per infusion) which account for approximately 0.5-3% of the T cells in the blood of an adult person. The increase in CD4⁺ T helper lymphocytes in the blood is several folds increased for all of the five patients, and the fast increase in T_{H} cells is not only due to the addition of the infused T_{H} cells, but might also be due to an endogenous T_{H} cell release from the bone marrow to the blood. The endogenous T_{H} cell release is the potential lifesaving physiological response we want to induce in the SurviveVirus treated patients, triggered by infusion of the novel T cell immunotherapy.

FIG. 4: Number T cells (CD3+), T helper cells (CD4+) and cytotoxic T cells (CD8+) in the composition of SurviveVirus produced with autologous and allogeneic DC's respectively. For more detail see also table 3.

FIG. 5: Level of interferon-γ (IFN-γ) produced by SurviveVirus cells. The level of IFN-γ in pg/mio cells were measured in the cell media when SurviveVirus cells were harvested day 7. There is no difference in the IFN-γ level whether the batch is produced with Allo- or AutoDC's, and in general is the IFN-γ level low. The average IFN-γ is 188.3 (+/−) 61.5 pg/mio when alloDC's are applied and 186.6 (+/−) 98.9 pg/mio cells respectively.

### DETAILED DISCLOSURE OF THE INVENTION

### Definitions

“Survivirus cells”, “Survivirus therapy” and “Survivirus process” generally relates to the cells, compositions, and processes of the invention disclosed herein. Likewise, the ALECSAT designation refers to the cells, compositions, and processes that are the subject of Kirkin et al. 2018 as well as WO 2020/208054.

“Allogeneic cells” are cells that—relative to an individual—have a different genotype than the individual's own (autologous) cells, and thus refers to genetic differences among individuals of the same species. A preferred from of allogeneic dendritic cells used in the present in the invention are those that exhibit a different HLA phenotype than the lymphocytes with which they are co-cultured.

An “autologous cell” is a cell derived from the individual to whom it is administered.

“Mononuclear cells” (also termed as “peripheral blood mononuclear cells”, abbreviated PBMC) denotes any cells of peripheral blood that have a rounded nucleus. The two main types of mononuclear cells are lymphocytes and monocytes, of which the latter have the ability to differentiate into macrophages and dendritic cells.

“Mature dendritic cells” (mature DCs) are in the present context dendritic cells that are obtainable by culturing monocytes under conditions described herein and which—in contrast to immature dendritic cells—have a high potential for T-cell activation. These mature dendritic cells, which are obtained by plating and culturing adhering monocytes, subsequently treating with IL-4 and GM-CSF to differentiate the monocytes into immature DCs and thereafter treating the immature DCs with TNF-alpha, IL-1β, IL-6, and prostaglandin E2, are not loaded with antigen.

“CD4⁺ lymphocytes”, “CD4⁺ cells” or “T_{H}” (the terms are used interchangeably herein) refer to lymphocytes of the T-helper subset. Among their functions are stimulation of B-cells and they also play an important role in the activation of CD8⁺ lymphocytes.

“CD8⁺ lymphocytes” or “CD8⁺ cells” or “cytotoxic T cells” (the terms are used interchangeably herein) refer to antigen specific lymphocytes that are capable of recognizing and killing cells that display MHC class I restricted T-cell epitopes.

“Natural killer cells” or “NK cells” or “NK lymphocytes” are antigen unspecific lymphocytes, which form part of the fast-reacting innate immune system, and which, as is the case of cytotoxic T cells, have the ability to kill cells. NK cells have a preferential ability to target cells that do not express MHC class I molecules.

The expression “increasing the CD4⁺/CD8⁺ ratio” is in the present context meant to indicate that a lymphocyte population that has been co-cultured with mature DCs as taught herein provides for a preferential expansion of the CD4⁺ subset of lymphocytes.

### SPECIFIC EMBODIMENTS OF THE INVENTION

The 1^{st} aspect of the invention relates—as indicated above—to a method for preparation of a composition of activated human CD4⁺ T helper cells and a lower proportion of CD8⁺ and natural killer (NK) lymphocytes.

In some embodiments of the first aspect of the invention, wherein the T lymphocytes from step c are analysed for indicators of phenotypic alteration. Such typing serves to characterize the cells obtained by the process but also as quality assurance that the cells have indeed changed their phenotype into a proliferating and activated phenotype. As a consequence, the first aspect of the invention entails embodiments, wherein the T lymphocytes are harvested and optionally isolated in step c when they exhibit proliferation (and/or activation) characteristics.

While step c might include a step of isolating the activated T-cells this is not normally a necessity. As Shown in the example below, only a minor portion of the mature allogeneic human dendritic cells of the co-culture are present at the conclusion of the co-culture, meaning that the harvested cells can be used in therapy without the activated and proliferating T-cells need by separated from these few remaining dendritic cells.

In order to further ensure that the lymphocytes are activated as part of the process, some important embodiments entail that an effective amount of IL-2 is added at least once during step b; typically, IL-2 is added when supplementing with fresh growth media during the co-culture process. As demonstrated herein, a suitable effective amount of IL-2 is added when supplementing with IL-2 at 25-60 IU per ml (for instance about 26 or 27 or 28 or 29 or 30 or 31 or 32 or 33 or 34 or 35 or 36 or 37 or 38 or 39 or 40 or 41 or 42 or 43 or 44 or 45 or 46 or 47 or 48 or 49 or 50 or 51 or 52 or 53 or 54 or 55 or 56 or 57 or 58 or 59 or 60 IU/ml).

In order to avoid or at least minimize the danger of adverse immunologic reactions when administering the cells obtained in step c, it is preferred that the T lymphocytes are harvested and optionally isolated in step c when substantially all allogeneic human dendritic cells are killed in the co-culture, or at least when they constitute an insignificant fraction of the end product. This is achieved if <0.1% of the final cell preparation is constituted by allogeneic human dendritic cells, even though fewer allogeneic human dendritic cells are preferred: e.g. <0.09%, <0.08%, <0.07%, <0.06%, <0.05%, <0.04%, and <0.03%. In particular, levels of about 0.02% are acceptable, even lower amounts are also preferred.

In embodiments of the first aspect, the ratio between CD4⁺ and CD8⁺ cells in the T-lymphocytes harvested in step c is preferably >1, such as >1.1, >1.2, >1.3, >1.4, and >1.5, thus mirroring the natural and normal ratio between CD4⁺ and CD8⁺ cells.

As detailed herein, the while it is possible to prepare the mature allogeneic mature dendritic cells immediately before the missing the autologous mononuclear cells, it is advantageous to prepare these cells in advance and keep them as a pre-prepared stock, which often will be cryopreserved (typically by employing methods and means well known for the skilled person), which allows easy application by thawing them when needed for step a.

In the co-culture step, the ratio between the mature allogeneic dendritic cells and the mononuclear cells is typically between 1:5 and 1:20 at the time of mixing in step a, such as between 1:9 and 1:11, and preferably about 1:10, cf. the examples.

While is it possible to use PBMCs as the mononuclear cells in step a, it is advantageous that the mononuclear cells in step a are monocyte depleted mononuclear cells. Depletion of monocytes is routinely done by methods generally applicable for the skilled person.

The preparation of the mature allogeneic human dendritic cells generally follows the teaching of Kirkin et al. 2018 and WO 2020/208054 but applied on donor cells. In general, it is preferred that the allogeneic human dendritic cells are genetically unmodified and derived from a human donor, which is to mean that not genetic engineering steps are undertaken to modify the cells, which hence have a native genotype. Hence the allogeneic dendritic cells are preferably prepared by culturing monocytes obtained from a human donor under conditions that facilitate maturation of dendritic cells. Such a method typically entails addition, during the course of culture, of granulocyte macrophage colony stimulating factor (GM-CSF) as well as Interleukin 4 (IL-4) and/or Interleukin 12 (IL-13), and optionally Interleukin 1β (IL-1β), Interleukin 6 (IL-6), Tumour Necrosis Factor α (TNF-α), and prostaglandin E2 (PGE2); typically, the process has a duration of about 6 days. Also the allogeneic dendritic cells are—when mixed with the mononuclear cells in step a—unloaded with antigen and they are also non-irradiated.

In an interesting embodiment, at least a fraction of harvested and optionally isolated T-lymphocytes is stored (e.g. cryopreserved) for later use after step c. This provides for the possibility to provide several doses to the autologous patient at different time points (if there are enough cells for this purpose).

### Further Description of the First Aspect

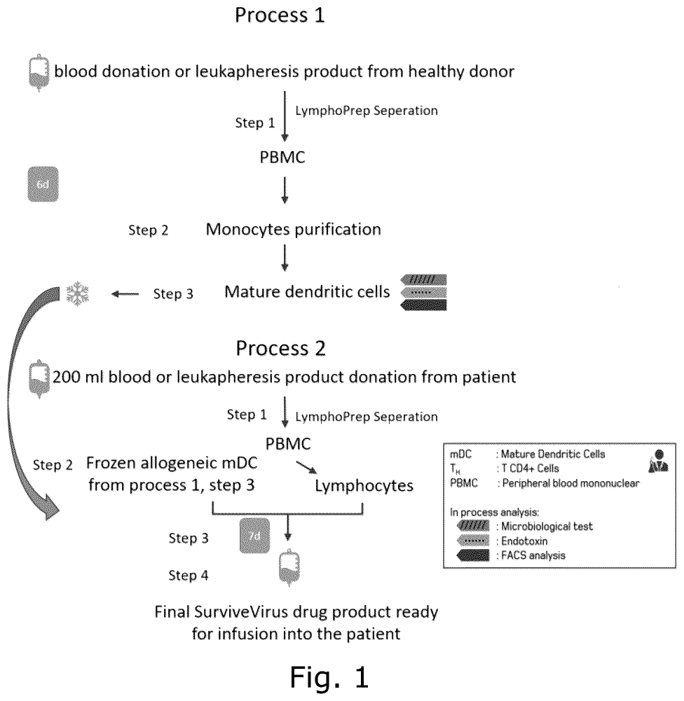

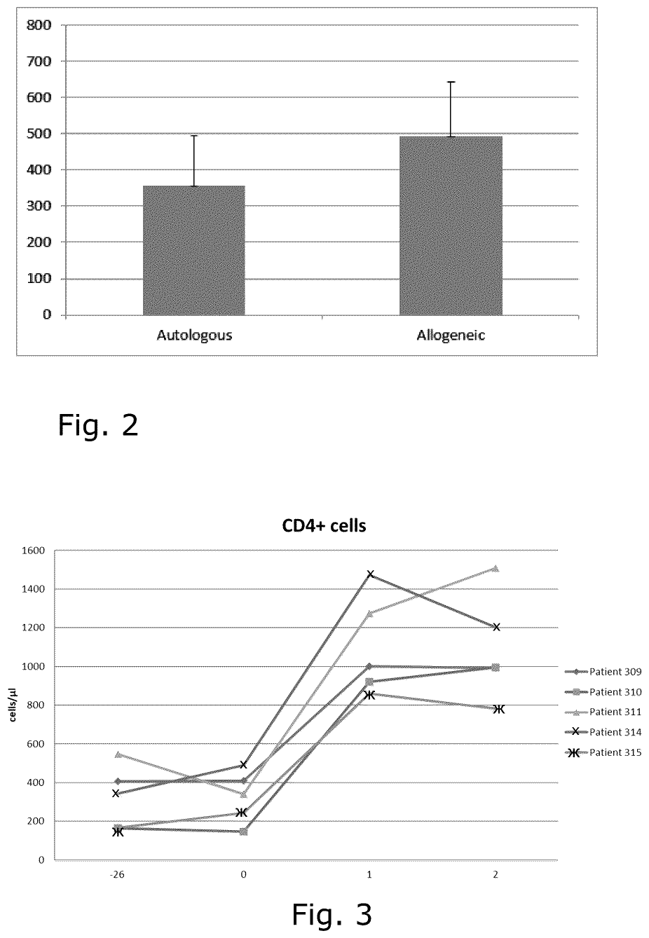

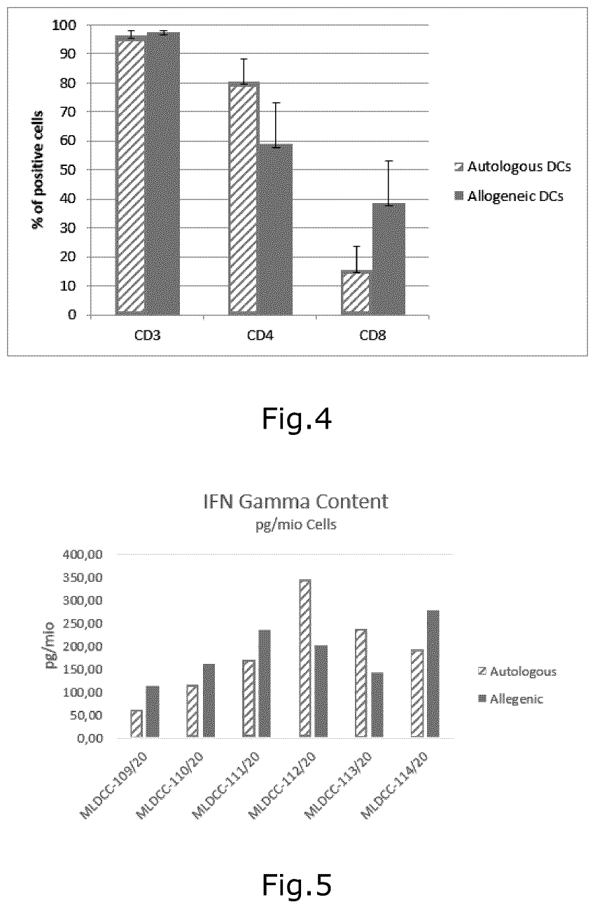

The overall process of the 1^{st} aspect is effectively divided into two processes (FIG. 1). Process 1 describes the 6-day long production process generating mature dendritic cells from the healthy donor monocytes.
1) Separation of peripheral blood mononuclear cells (PBMC) from donor blood or leukapheresis product
2) Monocyte purification
3) Generation of allogeneic monocyte derived mature dendritic cells by culturing a portion of the monocyte-enriched fraction of allogeneic cells, under conditions that facilitate maturation of mature dendritic cells

Process 2, which is the method of the first aspect takes 7 days if frozen or otherwise preserved allogeneic mDC's is produced in advance.
1) Separation of peripheral blood mononuclear cells (PBMC) from patient blood or leukapheresis product.
2) Mixing of allogeneic mDC's from process 1 and autologous lymphocytes (process 2, step 1), typically in a 1:10 ratio, in cell culture.
3) The mDC's induce activation and exponential growth of the CD4⁺ and CD8⁺-enriched lymphocytes.
4) The activated T cells are harvested, analysed and infused into the patient.

It will hence be clear that the monocytes/dendritic cells origin from a donor different from the subject from whom the autologous T-cells are obtained; the mature dendritic cells are allogeneic. The lymphocytes derive from the subject/patient and hence are isogeneic of origin. When the cell immunotherapy is infused into the patient 7 days post blood donation, the immunotherapy will consist of about 99.98% autologous cells since approximately 90% of the allogeneic dendritic cells are killed in the cell culture (see Table 2 below). The purpose of the allogeneic dendritic cells is to stimulate the proliferation and activation of the T cells.

The method is particularly useful for preparation of cells for use in personalised adoptive immunotherapy, where a patient's own T lymphocytes are activated and cultured to large numbers with the ability to stimulate endogenous T helper cells release to the blood in the patient.

The addition of the allogeneic DC's to the co-culture with the lymphocytes is a hallmark of the present invention.

Process 1, Step 1 (FIG. 1) describes separation of monocytes from PBMCs. Step 2, the monocytes are purified on a column in order to remove donor lymphocytes. Step 3, the allogeneic monocyte fraction is differentiated into mature dendritic cells according to the method for preparing mature DCs from monocytes in culture; this method include addition, during the course of culture, of 800 IU/ml granulocyte-macrophage colony stimulating factor (GM-CSF), 400 IU/ml Interleukin 4 (IL-4) to obtain immature DCs, followed by addition of 10 ng/ml TNFα 10 ng/ml Interleukin 1β (IL-1β), 1000 IU/ml Interleukin 6 (IL-6), and 0.1-1 μg/ml prostaglandin E2 (PGE2). The mDC's from the donor are kept frozen until they are used in process 2.

Process 2 (FIG. 1) Step 1, autologous lymphocytes are separated from PBMC. Step 2, A fraction of allogeneic mDC's is used for the 1:10 co-culture with the autologous lymphocytes.

Steps 3-4 are generally carried out as disclosed in WO 2008/081035A1 with the exception of the addition of allogeneic mature DC's as feeder cells in step 2, which is disclosed herein.

In all culturing steps, it has been found that IL-2 advantageously can be applied in concentrations 25-100 IU/ml, cf. the examples.

In all of aspects 1-2 of the invention, the last culture step is typically followed by recovery of all the cells including the activated CD4⁺, CD8⁺ and NK lymphocytes. These are then typically subsequently preserved for later use in therapy or they are used directly in the patient from which the cells are derived.

The method of the 1^{st} aspect of the invention and the embodiments described above has shown a remarkable ability to activate T-lymphocytes and render them proliferating. As shown in Table 4 in the examples, all or almost all T-cells obtained from the mixed cultures with allogeneic dendritic cells were shown to proliferate. Hence, in embodiments of the 1^{st} aspect of the invention, co-culturing in step b provides that at least 75% of CD4⁺ and CD8⁺ T lymphocytes in the mixed culture proliferate at the time of harvest in step c. However, this number is typically considerably higher, such as at least 80%, such as at least 85%, at least 87.5%, at least 90%, at least 92.5%, at least 95%, at 97.5%, at least 98%, and at least 99%.

### 2^{nd} and 3^{rd} Aspect of the Invention

As indicated above, the methods of the first aspect of the invention provides an improved composition of cells that is useful in adoptive immunotherapy which shall re-establish normal T_{H} cell number and function, in particular of the patient from whom the cells are originally derived, i.e. where the effector cells are autologous. The 2^{nd} aspect hence relates to a method for treatment of a patient, comprising of administering a composition of cells prepared as set forth above under 1^{st} aspect. One attractive feature of the present invention consists of the discovery of the effect of infusion of activate T cells into patients with lymphocytopenia as shown in FIG. 3 and the development of the novel immunotherapy SurviveVirus which can generate enough activated T cells to trigger the endogenous release of T_{H} cells within only 7 days. SurviveVirus can therefore be used to treat viral diseases such as COVID-19, which sometimes progresses very fast.

As mentioned above a related method instead entails that the composition is administered to an individual where the HLA type of the lymphocytes administered matches the HLA type of the recipient. Importantly, other relevant antigens (such as blood group antigens) between donor and recipient should also match in order to avoid graft versus host reactions as a consequence of administering the lymphocytes.

The treatment methods of aspects 2 and 3 are in particular useful when devising therapy for diseases that are known to cause acute lymphocytopenia. For instance, lymphocytopenia associated with or caused by an infectious agent selected from the group consisting of SARS-Cov, SARS-CoV-2, MERS-CoV, influenza virus, hepatitis virus and HIV are of particular relevance for these treatments. This means that the wherein the lymphocytopenia is associated with or caused by a disease or syndrome selected from the group consisting of SARS, MERS, Covid-19, AIDS, hepatitis, tuberculosis, and typhoid fever are particular useful target for the treatment.

The administration is normally via the intravenous route. The cells are conveniently suspended in an aqueous electrolyte-containing liquid used for intravenous infusion supplemented with autologous plasma or serum. Good results have been obtained with use of the isotonic infusion liquid Plasmalyte® (Baxter) supplemented with 1% (v/v) autologous serum.

Dosage of Medication in the 2^{nd} and 3^{rd} Aspect.

Infusion of SurviveVirus Cells.

SurviveVirus cells are manufactured from donor cells obtained from the blood bank (FIG. 1, table 1). It is shown, that the proliferation of the T cells is higher if allogeneic DC's are applied compared to autologous DC's. The presently presented protocol describes how 10 million non-activated lymphocytes can be expanded to 493 million activated large lymphocytes within 7 days in cell culture (see table 1 and FIG. 2). A patient will donate 200 ml blood and it will be possible to obtain 50-100 million lymphocytes, which is 5-10 times more cells than used in the present experiments. This results in the number of cells infused back to the patient potentially being 5-10 times higher than the experimental data presented in the table 1.

Infusion of an average of 68 million ALECSAT cells into Glioblastoma multiforme patients increased their T_{H} cell number per microliter of blood from approximately 200-400 to 800-1500. Less than 400 T_{H} cell/μl of blood is considered severe lymphocytopenia (FIG. 3). Therefore, the SurviveVirus protocol is designed to manufacture the same or even higher number of T-cells with a similar CD4/CD8 ratio as seen in the ALECSAT anti-cancer immunotherapy (Kirkin et al. 2018), but within a reduced period.

The SurviveVirus cell infusion has to be given within 7 days of vein to vein period in case of COVID-19 treatment, because the development of the disease in serious cases is so rapid. Some COVID-19 patients' condition is worsened already 7 days post their hospitalization, and they are transferred to intensive care. The SurviveVirus cell infusion can be delivered as early as day 7 post hospitalization; the activated T cells can in this stage help to regenerate the damaged immune defense in the patient, in order to control the infection and assist in saving the patients' lives. The infused T cells are intended to induce the endogenous T_{H} cell release which has observed in Glioblastoma multiforme patients (FIG. 3). The endogenous release of T_{H} cells brings the patients T_{H} cell number back to a normal level, which will help regulate the immune response in order to control the SARS-CoV2 infection.

### Example 1

Production of Expanded Population of Proliferating, Activated T-Lymphocytes.

In the following is provided a step-by-step protocol for use of allogeneic human dendritic cells in a protocol for T-cell expansion. The allogeneic DC's can be produced in advance, so the manufacturing time starts when the allogeneic DC's are mixed with the patient's lymphocytes (day 0). 6 days are used to produce the allogeneic DC's.

Generation of Mature Dendritic Cells from Blood of Allogeneic Donor.

The starting material is a source of allogeneic monocytes from a donor and autologous lymphocytes from the patient. The cells can be obtained from blood or a leukapheresis product. The protocol can be adjusted to the obtained number of lymphocytes from the patient. In this protocol it is preferable that lymphocytes and mDC's are mixed in a 10:1 ratio; all other reagents can be adjusted to the number of cells.

In the present example we have applied buffy coats obtained from the local Danish Blood Bank.

Upon arrival, blood (about 60 ml) was diluted with 60 ml of Ca and Mg free Dulbecco's Phosphate Buffered Saline (DPBS, Product No. BE17-512F, Cambrex, Belgium), and approximately 30 ml were layered on 15 ml of Lymphoprep® (Product No. 1053980, AXIS-SHIELD PoC AS, Norway) in four 50 ml tubes. After the first centrifugation at 200 G, 20 min, 20° C., 15-20 ml of the upper layer of plasma (so-called platelet rich plasma, PRP) were collected to a separate tube, and used for the preparation of serum. For this, CaCl₂) was added to a concentration of 25 mM, and after mixing, the plasma was transferred to a T225 flask (Nunc, Denmark), and placed in a CO₂-incubator. The flask was left in the CO₂-incubator until the next day. Centrifugation of tubes with Lymphoprep® was continued at 460 G, 20 min, 20° C. After termination of centrifugation, mononuclear cells were collected from the interface between Lymphoprep® and plasma to tubes with 25 ml of cold PBS-EDTA (Cambrex) and washed three times with cold PBS-EDTA by centrifugation, first at 300 G, then two times at 250 G, each time for 12 min at 4° C. After the last wash, cells were re-suspended in 30 ml of cold Ca and Mg free DPBS, and counted using a Moxi counter. Generation of dendritic cells (DCs) was performed in T225 tissue culture flasks pre-treated with 30 ml of 5% human AB serum and 50 U/ml of heparin in RPMI 1640. After removal of pre-treatment medium, 30 ml of a cell suspension containing 10×10⁷ PBMCs in AIM-V medium was added. After 30 min of incubation at 37° C., non-adherent lymphocytes were collected and discarded, whereas adherent monocytes were rinsed twice with pre-warmed RPMI 1640 medium and further cultured in 30 ml of AIM-V medium.

The T225 flask with the clotted plasma was transferred to a refrigerator and placed in an inclined position, with the clotted plasma down, and after 90-120 minutes, serum was transferred to a 50 ml tube, and transferred to a −20° C. freezer.

At day 1, a tube with the frozen autologous serum was transferred to the refrigerator (4° C.).

Tubes with the thawed serum were centrifuged at 2000 G, 15 min, 20° C., and the supernatant was transferred to a new 50 ml tube. This serum (termed “plasma-derived serum”) was stored at 4° C.

GM-CSF and IL-4 (both from Gentaur, Belgium, or CellGenix, Germany) were added to the flask with monocytes to final concentrations of 800 IU/ml and 500 IU/ml, respectively, at days 1 and 3.

At day 4, IL-18, IL-6, TNF-α (all from Gentaur), and PGE2 (Sigma) were added to final concentrations of 10 ng/ml, 1000 IU/ml, 10 ng/ml and 0.1-1 μg/ml, respectively, in 10 ml of AIM-V medium. Dendritic cells were harvested at day 6, counted, and frozen in aliquots of 3×10⁶ in freezing medium consisting of AIM-V medium (45%), autologous plasma-derived serum (45%) and 10% DMSO. Cell were kept either at −80° C. freezer, or in vapour phase of liquid nitrogen.

Generation of Allogeneic DC-Induced Activated Lymphocytes.

Day 0

Non-adherent monocyte-depleted lymphocytes were generated as described above for day 0 of generation of dendritic cells.

Frozen allogeneic non-adherent dendritic cells are thawed, counted and mixed with the fresh lymphocytes in a 1:10 ratio. After centrifugation, the mixture was re-suspended in 20 ml of lymphocyte medium consisting of AIM-V medium (Gibco, Invitrogen) and 2% autologous plasma derived serum, and placed T75 flask to side position.

Day 1

IL-2 (Gentaur) was added in 1 ml of AIM-V medium at final concentration of 50 IU/ml.

Day 3

20 ml of fresh lymphocyte medium supplemented with IL-2 (50 Wimp were added to the flask, and the flask was place to flat position.

Day 5

Cell culture was expanded by adding new medium with IL-2 and transfer to larger flask(s).

Day 7

The cell suspension was harvested, counted and analysed for phenotype by FACS analysis.

Results

In summary, an addition of allogeneic mature dendritic cells to the lymphocyte culture increases the proliferation rate of the T cells (FIG. 2 and Table 1).

TABLE 1

| | Lymphocytes (mio.) | Lymphocytes (mio.) |
|---|---|---|
| | (co-culture with | (co-culture with |
| Day | Autologous DCs) | Allogeneic DCs |
| 0 | 10.0 | 10.0 |
| 7 | 356.3 | 492.8 |

The table shows the cumulative number of T-lymhocytes in mixed cultures with autologous (n=6) and allogeneic (n=6) DCs respectively.

TABLE 2

| | | Number of | Number of | Percent |
|---|---|---|---|---|
| | FACS CD83 + | cells in | AlloDC's | AlloDC's |
| | HLA-2A+ | total batch | left | left |
| Batch 109 | 1/5,000 cells | 448 mio | 89,600 | 8.96% |
| Batch 111 | 1/5,000 cells | 512 mio | 102,400 | 10.20% |

An investigation of a number of preparations with respect to CD phenotype was also made. The table shows the phenotypic composition of SurviveVirus drug product produced with autologous and allogeneic DC's respectively. There can be some patient-to-patient variation in the cell composition and final cell number due to individual variation between patients and the donor DC's (Table 3):

TABLE 3
Autologous DC

| | Donor | Donor | Donor | Donor | Donor | Donor | | |
|---|---|---|---|---|---|---|---|---|
| CD markers | 109 | 110 | 111 | 112 | 113 | 114 | Average | SD |
| CD3 | 97.7 | 98 | 96 | 97.3 | 95.3 | 93.3 | 96.3 | 1.63 |
| CD4+CD8− | 79.7 | 81.9 | 81.8 | 64.8 | 88.2 | 86.7 | 80.5 | 7.62 |
| CD8+CD4− | 17.5 | 15.8 | 13.6 | 31.5 | 7.7 | 7.8 | 15.7 | 8.00 |

Allogeneic DCs

| | Donor | Donor | Donor | Donor | Donor | Donor | | |
|---|---|---|---|---|---|---|---|---|
| CD markers | 109/20 | 110/20 | 111/20 | 112/20 | 113/20 | 114/20 | Average | SD |
| CD3 | 96.6 | 98.4 | 97.7 | 97.9 | 96.6 | 97.4 | 97.4 | 0.7 |
| CD4+CD8− | 75.3 | 53.7 | 59.5 | 30.8 | 71.5 | 61.9 | 58.8 | 14.5 |
| CD8+CD4− | 22.0 | 43.4 | 36.9 | 67.6 | 26.2 | 34.9 | 38.5 | 14.8 |

Further the naïve T_{H} cells harvested from the patients' blood are getting activated by the presence of the mDC's and turned into activated T_{H} cells, which is seen as a significant increase in diameter of practically all T cells (table 4).

TABLE 4

| Lymphocyte diameter | Lymphocyte diameter |
|---|---|
| day 0 | day 7 (final SurviveVirus) |
| 8.5 μM | 12.4 μM |

This modification derived from steps in the existing ALECSAT technology leads to generation of T_{H} cells, which can be harvested for therapeutic purpose already after 7 days from withdrawing blood from the patient. This significantly increased the therapeutic potential against fast progressing viral diseases. The present invention takes advantage of the discovery of the effect of using allogeneic DC's in order to significantly reduce the time needed for manufacturing a batch of the immunotherapeutic SurviveVirus cells. ALECSAT is an immunotherapy designed for targeting cancer cells. The present immunotherapy, which aims at replenishing and/or can be manufactured 19 days faster than ALECSAT and can therefore be applied against fast progressing viral diseases causing lymphocytopenia where ALECSAT cells has proven their beneficial effect.

Clinical Relevance

There is a risk that the infusion of activated lymphocytes can induce further cytokine release syndrome, as it was shown after infusion of CAR-T cells (Borrega et al., 2019). Considering the fact that cytokine release syndrome is already seen in some COVID-19 patients and is probably associated with organ damage, the infusion of DC/IL-2 activated lymphocytes could potentially further enhance cytokine release syndrome and consequently induce more organ damage. Due to measurements of the cytokine profile and level of cytokines secreted by the cells prepared according to the present invention, the risk of such enhancement is however considered very low.

First of all, cytokine release syndrome seen after infusion of CAR-T cells is associated with production of cytokines after antigen-specific recognition of tumour cells. On the other hand, infusion of virus-specific lymphocytes usually do not induce cytokine release syndrome. The principal difference between CAR-T cells and virus-specific cells is the nature of their antigen receptor. In the CAR-T technology, lymphocytes are polyclonally activated by CD3/CD28 antibodies and transfected with chimeric antigen receptor (CAR) consisting of Fab fragment of antigen-specific antibody and one of the chains of T cell receptor antigen complex. Additionally, intracellular fragments of costimulatory molecules, either CD28, or 4-1BB, or both, are included. This makes the resultant CAR-T molecule highly sensitive to antigen stimulation, leading to hyper-activation of T lymphocytes and enhanced production of cytokines. However, in the case of virus specific T cells, natural T cell receptors are involved in antigen recognition, leading to elimination of virus infected cells without production of large amounts of cytokines. This may explain the lack of cytokine release syndrome after adoptive immunotherapy of virus-specific T lymphocytes. In case of DC/IL-2 activated lymphocytes produced according to the present invention, cytokine release syndrome should not be expected first of all due to lack of an antigen recognition process. Rather, the infusion is expected to induce restoration of normal level and functional activity of endogenous lymphocytes, leading to generation of virus-specific lymphocytes from these endogenous precursors. Due to employment of natural, unmodified T cell receptors in the antigen recognition process, no cytokine release syndrome is expected to take place in the patient.

### LIST OF REFERENCES

Bermejo-Martin J F, Almansa R, Menendez R, Mendez R, Kelvin D J, Torres A. Lymphopenic community acquired pneumonia as signature of severe COVID-19 infection: Lymphopenia in severe COVID-19 infection. J Infect 2020 Mar. 4.
Boonnak K, Vogel L, Feldmann F, Feldmann H, Legge K L, Subbarao K. Lymphopenia associated with highly virulent H5N1 virus infection due to plasmacytoid dendritic cell-mediated apoptosis oft cells. J immunol. 2014 jun 15; 192(12):5906-12. Doi: 10.4049/jimmunol.1302992.
Borrega J G et al., 2019. In the eye of the storm: immune-mediated toxicities associated with CAR-T cell therapy. Hemasphere 2019.
Brites C et al 2000. A randomized, placebo-controlled trial of granulocyte-macrophage colony-stimulating factor and nucleoside analogue therapy in AIDS. J Infect Dis. 2000 November; 182(5):1531-5.
Dudley M E, Wunderlich J R, Shelton T E, Even J, Rosenberg S A. Generation of tumor-infiltrating lymphocyte cultures for use in adoptive transfer therapy for melanoma patients. J Immunother. 2003 July-August; 26(4):332-42. doi: 10.1097/00002371-200307000-00005. PMID: 12843795; PMCID: PMC2305721.
Grupp S A, Prak E L, Boyer J, McDonald K R, Shusterman S, Thompson E, et al. Adoptive transfer of autologous T cells improves T-cell repertoire diversity and long-term B-cell function in pediatric patients with neuroblastoma. Clin Cancer Res 2012 Dec. 15; 18(24):6732-41. Laport et al, 2003
Kirkin A F, Dzhandzhugazyan K N, Guldberg P, Fang J J, Andersen R S, Dahl C, Mortensen J, Lundby T, Wagner A, Law I, Broholm H, Madsen L, Lundell-Ek C, Gjerstorff M F, Ditzel H J, Jensen M R, Fischer W. Adoptive cancer immunotherapy using DNA-demethylated T helper cells as antigen-presenting cells. Nat Commun. 2018 Mar. 6; 9(1):785. doi: 10.1038/s41467-018-03217-9. PMID: 29511178; PMCID: PMC5840134.
Kumar B V, Connors T J, Farber D L. Human t cell development, localization, and function throughout life. Immunity. 2018 Feb. 20; 48(2):202-213. Doi: 10.1016/j.immuni.2018.01.007.
Laport G G, 2003. Adoptive transfer of costimulated T cells induces lymphocytosis in patients with relapsed/refractory non-Hodgkin lymphoma following CD34+-selected hematopoietic cell transplantation. Blood-2003-01-0095
Laurell et al. 2017. Intratumorally injected pro-inflammatory allogeneic dendritic cells as immune enhancers: a first-in-human study in unfavourable risk patients with metastatic renal cell carcinoma. Journal for ImmunoTherapy of Cancer volume 5, Article number: 52 (2017).
Laux I, Khoshnan A, Tindell C, Bae D, Zhu X, June C H, et al. Response differences between human CD4(+) and CD8(+) T-cells during CD28 costimulation: implications for immune cell-based therapies and studies related to the expansion of double-positive T-cells during aging. Clin Immunol 2000 September; 96(3):187-97.
Levine B L, Bernstein W B, Aronson N E, Schlienger K, Cotte J, Perfetto S, et al. Adoptive transfer of costimulated CD4⁺ T cells induces expansion of peripheral T cells and decreased CCR5 expression in HIV infection. Nat Med 2002 January; 8(1):47-53.
Loosdrecht et al. A novel allogeneic off-the-shelf dendritic cell vaccine for post-remission treatment of elderly patients with acute myeloid leukemia. Cancer Immunol Immunother. 2018 October; 67(10):1505-1518
National Heart, Lung, and Blood Institute's homepage: www.nhlbi.nih.gov/health-topics/lymphocytopenia
Zhang X, Tan Y, Ling Y et al. Viral and host factors related to the clinical outcome of covid-19. Nature. 2020 July; 583(7816):437-440. Doi: 10.1038/s41586-020-2355-0. Epub 2020 may 20. Pmid: 32434211.
Van den Bergh J. M. J. et al. Monocyte-derived dendritic cells with silenced PD-1 ligands and transpresenting interleukin-15 stimulate strong tumor-reactive T-cell expansion. Cancer Immunology Research 2017 jun; 5(8): 710-715. Doi 10.1158/2326-6066.CIR-16-0336.

1. A method of for preparation of a composition of human T-cells, said composition comprising proliferating and activated CD4⁺ and CD8⁺ cells, the method comprising
a) mixing mononuclear cells from a human subject with mature allogeneic human dendritic cells,
b) co-culturing the mixed cells from step a under conditions that stimulate proliferation of CD4⁺ and CD8⁺ T lymphocytes, whereby the lymphocyte number is increased and the lymphocyte phenotypes are altered,
c) harvesting and optionally isolating T lymphocytes from the co-culture no later than 7 days after step a.

2. The method of claim 1, wherein the T lymphocytes from step c are analysed for indicators of phenotypic alteration.

3. The method of claim 1 or 2, wherein the T lymphocytes are harvested and optionally isolated in step c when they exhibit proliferation characteristics.

4. The method according to any one of the preceding claims, wherein IL-2 is added at least once during step b.

5. The method according to any one of the preceding claims, wherein the T lymphocytes are harvested and optionally isolated in step c when substantially all allogeneic human dendritic cells are killed in the co-culture.

6. The method according to any one of the preceding claims, where the fraction of CD4⁺ cells in the end product obtained in step c is larger than the fraction CD8⁺ cells and larger than the fraction of NK cells.

7. The method according to any one of the preceding claims, wherein the ratio between CD4⁺ and CD8⁺ cells in the T-lymphocytes harvested in step c is >1, such as >1.1, >1.2, >1.3, >1.4, and >1.5.

8. The method according to any one of the preceding claims, wherein the mature allogeneic human dendritic cells are provided from a pre-prepared stock, preferably cryopreserved.

9. The method according to any one of the preceding claims, wherein the ratio between the mature allogeneic dendritic cells and the mononuclear cells is between 1:8 and 1:12 at the time of mixing in step a, such as between 1:9 and 1:11, and preferably about 1:10.

10. The method according to any one of the preceding claims, wherein the mononuclear cells in step a are monocyte depleted mononuclear cells.

11. The method according to any one of the preceding claims, wherein the mature allogeneic dendritic cells are genetically unmodified cells derived from a human donor.

12. The method according to any one of the preceding claims, wherein the mature allogeneic human dendritic cells are prepared by culturing monocytes obtained from a human donor under conditions that facilitate maturation of dendritic cells.

13. The method according to claim 12, wherein the culturing of monocytes includes addition, during the course of culture, of granulocyte macrophage colony stimulating factor (GM-CSF) as well as Interleukin 4 (IL-4) and/or Interleukin 12 (IL-13), and optionally Interleukin 1β (IL-1β), Interleukin 6 (IL-6), Tumour Necrosis Factor α (TNF-α), and prostaglandin E2 (PGE2).

14. The method according to claim 12 or 13, wherein obtaining and culturing the monocytes has a duration of about 6 days.

15. The method according to any one of the preceding claims, wherein the allogeneic dendritic cells are unloaded with antigen and non-irradiated.

16. The method according to any one of the preceding claims, wherein co-culturing in step b provides that at least 75% of CD4⁺ and CD8⁺ T lymphocytes in the mixed culture proliferate at the time of harvest in step c.

17. The method according to claim 16, wherein at least 80%, such as at least 85%, at least 87.5%, at least 90%, at least 92.5%, at least 95%, at 97.5%, at least 98%, and at least 99% of CD4⁺ and CD8⁺ T lymphocytes in the mixed culture proliferate at the time of harvest in step c.

18. The method according to any one of the preceding claims wherein at least a fraction of harvested and optionally isolated T-lymphocytes are stored for later use after step c.

19. The method according to claim 18, wherein the T-lymphocytes are cryopreserved.

20. The method according to claim 18, wherein the T-lymphocytes are HLA-typed.

21. A method for supplementing a human subject with lymphocytes, such as in a treatment of lymphocytopenia in a human subject, the method comprising obtaining a sample comprising blood cells from the subject, isolating mononuclear cells from the sample and subsequently preparing a composition of human T-cells according to the method of any one of the preceding claims, wherein the mononuclear cells in step a are the mononuclear cells from the sample, and subsequently administering an effective amount of the T lymphocytes obtained from step c to the patient.

22. A method for treatment of lymphocytopenia in a human subject, the method comprising administering to said human subject T-lymphocytes obtained by the method according to any one of claims 1-20, wherein said T-lymphocytes matches the HLA phenotype of said human subject.

23. The method according to claim 21 or 22, wherein the lymphocytopenia is associated with or caused by an infectious agent selected from the group consisting of SARS-Cov, SARS-CoV-2, MERS-CoV, influenza virus, hepatitis virus and HIV.

24. The method according to any one of claims 21-23, wherein the lymphocytopenia is associated with or caused by a disease or syndrome selected from the group consisting of SARS, MERS, Covid-19, AIDS, hepatitis, tuberculosis, and typhoid fever.

25. A composition of proliferating and activated T-lymphocytes obtainable or obtained by the method according to any one of claims 1-20.

26. The composition according to claim 25 for use in supplementing lymphocytes to a patient or treating lymphocytopenia, preferably according to the method according to any one of claims 21-24.
